(19) United States
(12) Patent Application Publication
(10) Pub. No.: US 2023/0301895 A1
(43) Pub. Date: Sep. 28, 2023

(54) NAIL FORMULATIONS AND TREATMENT REGIMES

(71) Applicant: NOVABIOTICS LIMITED, Bridge of Don, Aberdeen (GB)

(72) Inventors: Deborah O’NEIL, Bridge of Don, Aberdeen (GB); Derry MERCER, Bridge of Don, Aberdeen (GB)

(73) Assignee: NOVABIOTICS LIMITED, Bridge of Don, Aberdeen (GB)

(21) Appl. No.: 18/004,134

(22) PCT Filed: Jul. 2, 2021
(86) PCT No.: PCT/GB2021/051685

§ 371 Date: Jan. 3, 2023

(30) Foreign Application Priority Data
Jul. 3, 2020 (GB) 2010268.7

(51) Int. Cl. A61K 8/65; A61K 8/86; A61K 8/67; A61Q 3/02; A61Q 17/00; A61K 8/64

(52) U.S. Cl. CPC A61K 8/65; A61K 8/64; A61K 8/673; A61K 8/86; A61Q 3/02; A61Q 17/005; A61K 2800/95

(57) ABSTRACT

The invention relates to aqueous formulations for topical nail application. The invention also relates to compositions for use in the treatment of a fungal nail infection, the composition being applied over a defined period.

### FIELD OF THE INVENTION

The present invention relates to an aqueous composition containing a protein which is suitable for topical application to a nail. The invention also relates to cosmetic use of the composition and use of the composition as a medicament including for the treatment of fungal nail infections. The invention also includes devices containing the composition, the devices aiding in applying the composition to the nail; and use of PEGs as film-forming agents in nail compositions containing proteins.

### BACKGROUND TO THE INVENTION

Formulating proteins into liquid compositions which can be easily applied to a nail and are therefore suitable for topical application; and maintains efficacy of the protein, is a complex process.

The present invention provides such compositions.

### STATEMENTS OF THE INVENTION

According to a first aspect there is provided an aqueous composition for topical nail application, the composition comprising:
a) a protein; and
b) polyethylene glycol (PEG) 500 - PEG 8000.

Advantageously, the use of an aqueous composition comprising PEG at these molecular weights (mwt) provides a composition which dries quickly following application to the nail and allows the formation of a film on the nail to form allowing the user to see the area of the nail the product has been applied to and as a barrier to protect the surface of the nail. Therefore, the composition is a film-forming composition with PEG as the film-forming agent.

The PEG may have an average molecular weight from 1000 and 6000, for example 1500 to 6000 such as 3000 to 6000 or 4000 to 6000. The composition may comprise between 10 and 80% v/v PEG, for example 40-60% v/v PEG.

The protein may be collagen and/or keratin. The composition may comprise a nail enhancer, for example biotin.

In a further aspect, the invention includes a cosmetic method for treatment of the nail, the method comprising applying the composition described above. After application, the composition may form a film on the nail.

The protein may alternatively be an antifungal cyclic peptide comprising 4-15 arginines subject to 0 or 1 substitutions in the peptide. That is, the composition is an antifungal aqueous composition for topical nail application.

The antifungal composition may additionally comprise any one or more of the following: a) a protease inhibitor, optionally a metallopeptidase inhibitor; b) keratin; c) collagen or d) a nail enhancer, for example biotin.

In a further aspect, there is provided an aqueous composition comprising:
a) an antifungal cyclic peptide comprising 4-15 arginines subject to 0 or 1 substitutions in the peptide; and
b) a protease inhibitor, optionally a metallopeptidase inhibitor.

The metallopeptidase inhibitor may be an endoprotease inhibitor, optionally
a) an inhibitor of the fungalysin family (M36); or
b) ethylenediaminetetraacetic acid (EDTA).

The cyclic peptide may be present at about 1% w/v to 20% w/v of the composition. The cyclic peptide may comprise 5 to 9 arginine residues. The antifungal cyclic peptide may be in an acid salt form, optionally acetate.

In a further aspect, a pharmaceutical composition is provided comprising any of the antifungal aqueous compositions described above.

In a further aspect, the antifungal composition is provided for use as a medicament.

The use may be for the treatment or prevention of a fungal nail infection wherein the composition is applied topically. The fungal nail infection may be caused by a dermatophyte. The composition may be applied daily, optionally over a period of 7-28 days.

In a further aspect, there is provided an antifungal cyclic peptide comprising 4-15 arginines subject to 0 or 1 substitutions in the peptide, for use in the treatment or prevention of fungal nail infection, wherein the composition is applied daily over a period from 7-28 days. The composition may dry to the surface of the nail over a period from 3 minutes to 35 minutes. The composition may be any of the aqueous compositions provided above. The treatment may be repeated every 3-12 months. The antifungal cyclic peptide may be in an acid salt form, optionally acetate. The peptide may be present at about 1% w/v to 20% w/v of the composition. The composition for use may be any of the compositions described above comprising antifungal cyclic peptides.

In a further aspect, a device is provided which is adapted to apply any of the compositions described. The device may be a nail pen.

In a further aspect, the use of PEG 500-8000 is provided as a film-forming agent in aqueous topical nail compositions comprising a protein.

### BRIEF DESCRIPTION OF THE DRAWINGS

The present invention will now be described by way of example only with reference to the accompanying Figures in which:

FIG. 1: Metabolic Activity of T. rubrum NCPF0118 Exposed to NP213 in Roehm Lacquer 1 and PEG4000 Formulation

FIG. 2: Metabolic Activity of T. rubrum NCPF0118 Exposed to NP213 in Roehm Lacquer 2 and PEG4000 Formulation

FIG. 3: Metabolic Activity of T. rubrum NCPF0118 Exposed to NP213 in Roehm Lacquer 3 and PEG4000 Formulation

FIG. 4: Metabolic Activity of T. rubrum NCPF0118 Exposed to NP213 in Roehm Lacquer 4 and PEG4000 Formulation

FIG. 5: Metabolic Activity of T. rubrum NCPF0118 Exposed to NP213 in Roehm Lacquer 5 and PEG4000 Formulation

FIG. 6: Metabolic Activity of T. rubrum NCPF0118 Exposed to NP213 in Anacor Lacquer C and PEG4000 Formulation

FIG. 7: Metabolic Activity of T. rubrum NCPF0118 Exposed to NP213 in 2d Lacquer and PEG4000 Formulation

FIG. 8 shows the average drying time on excised nail of different mwt PEG compositions at 50% PEG concentration with water and optionally with 10% peptide. 3 replicates were used in each case.

FIG. 9: Effect of the metalloprotease inhibitor, EDTA (100 µM), on the antifungal efficacy of Novexatin® (NP213) during growth of T. rubrum NCPF0118 on 0.75% (w/v) human nail for 216 h

FIG. 10: Effect of the metalloprotease inhibitor, EDTA (100 µM), on the antifungal efficacy of Novexatin® (NP213) during growth of T. interdigitale NCPF0335 on 0.75% (w/v) human nail for 216 h

FIG. 11: Effect of the metalloprotease inhibitor, EDTA, on the metabolic activity of T. rubrum NCPF0118 and T. interdigitale NCPF0335 after 168 h incubation

FIGS. 12 and 13: Effect of substituting a single Arginine residue for a Phenylalanine (F) or Lysine (K) residue on the activity of NP213 against T. rubrum NCPF0118 and T. interdigitale NCPF0335.

FIG. 14: Photograph of film made by the peptide and PEG combination.

FIG. 15: Effect of NP213 concentration (% w/v) and time (d) on antifungal efficacy of NP213 against T. rubrum NCPF0118

FIG. 16: Example of Nail applicator device: nail bottle and brush

FIG. 17: Example of Nail applicator device: nail pen

### DETAILED DESCRIPTION

### Composition

The composition is aqueous meaning it comprises water. The composition is a solution, i.e. the composition is a liquid. The composition may be particulate free meaning there are no undissolved particles, e.g. undissolved crystals, of the peptide in the composition. The skilled person would be able to determine if the solution was particulate free by for example centrifuging the solution and checking visually for particulates, e.g. crystals of the peptide, in the bottom of the centrifuged tube. Alternatively visual inspection as described in Example 3 may be used.

The composition is a topical composition. That is, the composition is suitable for topical application.

### Protein

By protein is meant a plurality of amino acid residues joined together by peptide bonds. The term protein encompasses longer lengths of amino acid residues as well as peptides of shorter lengths of amino acids.

The protein may be any which has efficacy in enhancing or improving the appearance of the nail. For example, a protein which strengthens the nail. For example, the protein may be keratin and/or collagen. The amino acid sequence of these proteins is known. The protein may be the full length protein collagen or keratin. Alternatively, the collagen or keratin may be a shortened or mutant version which retains all or part of the activity of the full length protein to increase the strength of the nail.

Or the protein may have therapeutic activity for medical treatment of the nail.

### Cosmetic Method

The composition may be used in a cosmetic method of treatment of the nail. By cosmetic is meant non-medical treatment to improve the appearance of the nail.

### Peptide

The protein may be a peptide. For example, a therapeutically active peptide. This includes a salt thereof, i.e. a pharmaceutically acceptable salt. The peptide may have activity against fungal infections of the nail, i.e. the peptide is an antifungal peptide.

The peptide may be a cyclic peptide. Methods for backbone cyclisation of peptides are known in the art. The cyclic peptide may comprise or consist of 4 to 15 amino acids, for example 4 to 14 amino acids; 4 to 9 amino acids, 4 to 8 or 4 to 7 amino acids. The peptide may comprise or consist of 4, 5, 6, 7, 8, 9, 10, 11, 12, 13, 14 or 15 amino acids. The peptide may consist of 7 amino acids. The peptide may be a polyarginine peptide. For example, cyclic R-R-R-R-R-R-R. This cyclic Arginine 7-mer peptide is referred to as NP213 in the examples below.

One of the residues of the cyclic peptide can be exchanged for another residue. Such a variant can have, for example, at least about 10%, 50%, 60%, 70%, 80%, 90% or the equivalent biological activity of the corresponding non-variant peptide when tested for example in vitro using the method explained in Example 6. Conservative amino acids are often utilised, i.e. substitutions of amino acids with similar chemical and physical properties. Hence, for example, conservative amino acid substitutions may involve exchanging arginine for lysine or isoleucine. Histidine and other cationic amino acids, including non-naturally occurring cationic amino acids, may also be substituted. The inventors have also shown exchanging arginine for phenylalanine to also result in a peptide with anti-fungal activity. After the substitutions are introduced, the variants are screened for biological activity, using for example, the method explained in Example 6.

The amino acids in the peptide may be D or L amino acids. For example, the amino acids in the peptide may be 90% or 95%, 98% or 99% L amino acids. The activity of the peptide is as a result of the cationic charge of the peptide which acts by disrupting and permeabilizing the fungal cytoplasmic membrane.

The definition of the peptide also includes known isomers (structural, stereo-, conformational & configurational), peptidomimetics, structural analogues of the above amino acids, and those modified either naturally (e.g. post-translational modification) or chemically, including, but not exclusively, phosphorylation, glycosylation, sulfonylation and/or hydroxylation.

The definition of the peptide also includes salt forms of the peptide. For example, an acid salt form. These include acetate, hydrochloride and trifluoroacetate/trifluoroacetic acid (TFA) salt forms. For example, NP213 (R-R-R-R-R-R-R) acetate. Additional acetic acid may also be added to the composition.

The peptides generally are synthetic peptides. The peptides may be isolated, purified peptides or variants thereof, which can be synthesised in vitro, for example, by a solid phase peptide synthetic method, by enzyme catalysed peptide synthesis or with the aid of recombinant DNA technology.

The composition may comprise 0.01-20% of the peptide. For example 1, 2, 3, 4, 5, 6, 7, 8, 9, 10, 11, 12, 13, 14, 15, 16, 17, 18, 19 or 20%. For example, 5-15%; or 8-12%. The percentage is weight to volume (w/v). That is, weight of the peptide in grams divided by the volume of the final solution in millilitres multiplied by 100. Typically, the peptide is prepared by first dissolving the peptide in water and then diluting the peptide and water solution in liquified PEG to the concentration required in the final solution.

Example peptides are as follows:
1) cyclic R-R-R-R-R-R-R
2) cyclic R-R-R-R-R-R-F
3) cyclic R-R-R-R-R-R-K

### PEG

As employed herein PEG is polyethylene glycol, a polymer of ethylene oxide. The molecular weight of the polymer is typically denoted by numbers following PEG, for example PEG8000 has a molecular weight of 8000 g/mol.

The PEG may be PEG 1000-6000. The PEG may be PEG 1000, PEG 1500, PEG 2000, PEG3000, PEG 3350, PEG4000, PEG 5000, PEG 6000, PEG 7000 or PEG 8000. That is PEG with an average molecular weight (mwt) of 4000 g/mol or 3000 g/mol respectively, or PEG of a higher average molecular weight, such as PEG6000 or PEG8000.

The concentration of PEG is % (v/v). If solid, the PEG is melted and then mixed as a solution with the cyclic peptide which is dissolved in H₂O. Lower molecular weight PEGs are liquid at room temperature, e.g. below PEG1000. PEG1000 and the heavier PEGs are typically all solid at room temperature (i.e. <30° C.).

The composition may comprise 10-80% of any of the above PEGs. For example, the composition may comprise 20-70% PEG or 30-60% PEG or 40-60 PEG. For example 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80% v/v. For example, 45-50% PEG v/v.

The composition may comprise up to about 60% v/v PEG, for example about 40% to 60% v/v PEG. In one embodiment the composition comprises 40% to 55% v/v PEG. In an alternative embodiment, the composition comprises 45% to 50% v/v PEG

The composition may comprise from 47.5 to about 55% v/v PEG4000 or about 47.5 to about 60% v/v PEG3000. The PEG may comprise approximately 50% v/v PEG4000. Alternatively, the PEG may comprise approximately 52.5% v/v PEG3000.

### Combinations of PEG and the Peptide

Example compositions include 2-20% peptide (w/v), for example poly-arginine 7-mer with up to 1 substitution, and 40-80% PEG 1000-PEG8000 (v/v). EDTA may be added to this combination at a concentration of at least 15 µM.

The composition may comprise 5-10% peptide (w/v), and 40-55% PEG 1000-8000 (v/v).

The composition may comprise 5-12.5% peptide (w/v), and 40-55% PEG 1000-6000 (v/v).

The composition may comprise 5-15% peptide (w/v), and 40-55% PEG 1000-3000 (v/v).

### Units

As an example of how to prepare a formulation: To prepare a 10% (w/v) NP213 solution in 50% (v/v) PEG4000, all materials used were pre-warmed to 37° C. in an incubator before starting the formulation work to ensure and facilitate accurate dispensing of liquified PEG. NP213 was prepared as an aqueous solution in sterile deionised water (18 MΩ purity) at a concentration of 400 mg/ml, filtered through a 0.22 µm syringe filter to ensure sterility, and pre-warmed to 37° C. immediately prior to use in formulation preparation. 20 g PEG4000 (solid at room temperature; melting point 53 - 58° C.) was melted in a glass beaker in a microwave oven on low power until a clear solution was obtained. At no point was the temperature of the PEG4000 allowed to exceed 70° C. Once liquified, 5 ml PEG4000 was transferred to a sterile glass universal bottle, placed in a 37° C. incubator and allowed to equilibrate to 37° C. for 30 - 60 min. To this was added 2.5 ml NP213 (400 mg/ml; 40% (w/v))and the volume was made up to 10 ml with pre-warmed sterile deionised water (18 MΩ purity). Solutions were stored at room temperature (18 - 20° C.) in the dark.

### Protease Inhibitor

The composition may comprise a protease inhibitor, optionally a metallopeptidase inhibitor. By protease inhibitor is meant a compound which inhibits the activity of a protease, i.e. a fungal protease.

The protease inhibitor may be a cysteine protease inhibitor, a serine protease inhibitor or an aspartyl protease inhibitor. Examples of cysteine protease inhibitors include antipain dihydrochloride, chymostatin, N-Ethylmaleimide, Leupeptin, α2-Macroglobulin. E-64 and Phenylmethanesulfonyl fluoride (PMSF). Examples of serine protease inhibitors include 4-(2-Aminoethyl)benzenesulfonyl fluoride (AEBSF/Pefabloc), Aprotinin, Leupeptin, serpins and Phenylmethanesulfonyl fluoride (PMSF). Examples of aspartyl protease inhibitors include Pepstatins, α2-Macroglobulin, Ritonavir, Indinavir, Zankiren,Aliskiren and LY-450139.

The concentration of the protease inhibitor may be 0.5 µM - 500 µM. For example 15 µM-500 µM. For example, 50, 100, 150, 200, 250, 300, 350, 400, 450 or 500 µM or any range within these concentrations.

The metallopeptidase inhibitor may be an endoprotease inhibitor, for example, an inhibitor of the fungalysin family (M36). For example, groups of metallopeptidase inhibitors include: Azapeptide inhibitors, Hydroxamate-containing inhibitors, Boron-containing inhibitors, Carboxylate-containing inhibitors, Small molecule inhibitors, Chelating inhibitors, Peptide inhibitors, Diazo inhibitors, DNA aptamer inhibitors, RNA aptamer inhibitors, Phosphorus-containing inhibitors, Haloketone inhibitors, Polyphenol inhibitors of metallopeptidases, Ketomethylene inhibitors, Thiol inhibitors, Thiirane inhibitors, Matrix metalloprotease (MMP) inhibitors, Selenium-containing inhibitors and Silanediol inhibitors or any combination thereof.

Individual examples within these groups include the following: EDTA (Ethylenediaminetetraacetic acid), EGTA (ethylene glycol-bis(β-aminoethyl ether)-N,N,N′,N′-tetraacetic acid), Bestatin, 2,2′-Bipyridyl 1,10-Phenanthroline monohydrate, Phosphoramidon disodium salt, Doxycycline hyclate, marimastat, batimastat, Galardin, alatrioprilat, anacardic acid, ebselen, glycoprilat, 4-methyl-1-(S)-({5-[(3-oxo-3,4-dihydro-2H-benzo[1,4]oxazin-6-ylmethyl)-carbamoyl]-pyrazolo[1,5-a]pyrimidine-7-carbonyl}-amino)-indan-5-carboxylic acid, namalide, purpurin, regasepin1, regasepin2, salvianolic acid B Triptolide, 2-benzyl-4-oxo-5,5,5-trifluoropentanoic acid, p-iodo-D-phenylalanine hydroxamate 4,4′-phosphinicobis(butane-1,3-dicarboxylic acid), alpha-lipoic acid, L-butaneboronic acid, glycolic acid, thiolutin, 2-benzyl-4-oxo-5,5,5-trifluoropentanoic acid, epigallocatechin-3-gallate Myricetin, hinokiflavone and thimerosal.

The concentration of the metallopeptidase inhibitor in the composition may be at least 15 µM. For example 15 µM-500 µM. For example, 50, 100, 150, 200, 250, 300, 350, 400, 450 or 500 µM or any range within these concentrations.

The inhibitor may be EDTA or EGTA and the concentration of the EDTA may be at least 15 µM. For example, 15-150 µM. The concentration of EDTA or EGTA in the composition may be at least 50 µM. For example 100 µM.

### Nail Enhancers and Other Additional Compounds

Other compounds can also be added to the compositions. For example, other compounds which enhance penetration of the proteins and peptides into the nail. Alternatively, compounds may be added which enhance the appearance of the nail (referred to as nail enhancers). The nail enhancer may be biotin or an optical brightener. The composition may further comprise a pigment and/or a perfume.

The composition may not comprise urea or organic solvents.

### Pharmaceutical Composition

The compositions have therapeutic utility. Therefore, the compositions may be pharmaceutical compositions which further comprise one or more pharmaceutically acceptable carriers, adjuvants, excipients, diluents, fillers, buffers, stabilisers, preservatives, lubricants, or other materials well known to those skilled in the art and optionally other therapeutic agents.

### Fungal Infection

The compositions described herein have utility as medicaments, in particular against fungal nail infections.

As employed herein a fungal infection is an infection caused by a fungus, typically a pathogenic fungal species such as a dermatophyte. For example, belonging to the genera Trichophyton, Microsporum or Epidermophyton, such as Trichophyton rubrum.

The fungus is generally a dermatophyte, such as those isolated from a tinea infection, such as a tinea unguium, tinea corporis, tinea capitis, tinea cruris, tinea faciae and tinea pedis infection. The dermatophyte may be an isolate of Trichophyton spp. The dermatophyte may be an isolate from the following genera; Trichophyton spp., Arthroderma spp., Microsporum spp., Lophophyton spp., Nannizzia spp., Epidermophyton spp., Paraphyton spp., Guarromyces spp. or Ctenomyces spp. The dermatophyte may be from the following species; T. benhamiae, T. bullosum, T. concentricum, T. equinum, T. eriotrephon, T. erinacei, T. interdigitale, T. mentagrophytes, T. quickeanum, T. rubrum, T. schoenleinii, T. simii, T. soudanense, T. tonsurans, T. verrucosum, T. violaceum, E. floccosum, N. aenygmaticum, N. corniculate, N. duboisii, N. fulva, N. gypsea, N. incurvata, N. nana, N. persicolor, N. praecox, P. cookei, P. cookiellum, P. mirabile, L. gallinae, M. audouinii, M. canis, M. ferrugineum, A. amazonicum, A. ciferrii, A. cuniculi, A. curreyi, A. erboreum, A. flavescens, A. gertleri, A. gloriae, A. insigulare, A. lenticulare, A. melis, A. multifidum, A. onychocola, A. phaseoliforme, A. quadrifidum, A. redellii, A. silverae, A. thuringiensis, A. tuberculatum, A. unicatum or A. verpertilii.

Alternatively, the fungus may be a yeast such as Candida spp., typically Candida albicans, Candida krusei, C. glabrata, C. guillermondii, C. famata, C. parapsilosis, C. tropicalis, C. sake, Malassezia furfur and Trichosporon spp.

According to a further aspect of the present invention the fungus may be a non-dermatophyte mould such as Acremonium spp (for example A. roseogriseum), Alternaria spp., Arthrographis kalrae, Aspergillus spp. (including A. flavus, A. fumigatus, A. terreus, A. ustus, A. sydowii, A. versicolor), Bipolaris spp., Botryodiplodia theobromae, Chrysosporium (Geomyces) pannorum, Cladosporium spp., Fusarium spp. (including F. oxysporum, F. proliferatum, F. solani), Geotrichium candidum, Nattrassia spp., Onychocola canadensis, Paecilomyces spp., Penicillium spp., Phyllostricta sydowii, Pyrenochaeta unguis-hominis, Scopulariopsis brevicaulis, Scytalidium spp. (including S. didmidiatum, S. hyalinum), Synchephalastrum racemosum, Trichoderma spp. and Ulocladium spp.

### Treatment

The composition comprising an antifungal cyclic peptide comprising 4-15 arginines subject to 0 or 1 substitutions in the peptide is useful in the treatment of fungal nail infections such as onychomycosis. Optionally, this composition is the aqueous composition comprising PEG and/or protease inhibitors and/or other additives as described above.

There is provided a method of treating a nail infection comprising the steps of applying a therapeutically effective amount of the composition described herein to a nail suspected of infection, spreading the composition over the surface of the nail, waiting for the composition to dry and optionally repeating the application, for example approximately 24 hours later, i.e. the treatment is at least once per day. The treatment may also be applied 2 or 3 times/day.

Typically nail treatments require use for up to 48 weeks or more before improvement i.e. improvement in appearance of nails and a reduction in fungal burden within the nail. By contrast, the cyclic peptide composition described above requires only 7-11 days before improvement is seen. Therefore, the treatment may be applied daily for 7-11 days or more, for example 7, 8, 9, 10, 11, 12, 13, 14, 15 or 16 days. Alternatively, the treatment may continue for severe infections up to 17, 18, 19, 21, 22, 23, 24, 25, 26, 27 or 28 days. The treatment may then stop. To prevent reoccurrence of the infection, the treatment may be applied every 3 months or every 6 months or 9 months or 12 months. For example, the treatment may be 7, 8, 9, 10, 11, 12, 13, 14, 15 or 16 days every 3 months, or 6 months or 9 months or 12 months. Or the treatment may be applied for 17, 18, 19, 20, 21, 22, 23, 24, 25, 26, 27 or 28 days every 3, 6, 9 or 12 months.

For example, the treatment may be daily treatment for 21 days, every 3-6 months. The treatment may also be applied every 9 or 12 months.

Dosage may be relatively high dosage to eliminate the infection in the first treatment. For example, 8-10% for 7-11 days or any of the treatment periods above.

Prevention of re-infection may be at a lower dose. For example, 1-7% daily for any of the treatment periods above, e.g. 7-11 days, every 3-6 months. Prevention may also continue by treating every 9 or 12 months.

Alternatively, all dosing may be at 2% w/v of the peptide. For example, the first treatment may be 2% w/v of the peptide applied at least once per day. This treatment may last 7-28 days. Prevention may then also be by administering with 2% w/v daily for 7-28 days every 3-6 months for subsequent applications (or every 9-12 months).

Alternatively, this dosing may be 3%-9%, for example, 3%, 4%, 5%, 6%, 7%, 8%, 9%.

The treatment is topical meaning application to the nail surface and surrounding skin. Topical application of the treatment means that the composition is applied directly to the accessible side (that is the exposed side away from the nail bed) of the nail and may optionally be applied to the free edge of the nail (the free edge being the part of the nail that extends beyond the nail bed).

As well as drying quickly, the PEG formulation allows the patient to see a film of the solution on the nail due to the presence of the PEG. This makes the patient aware of any areas missed and the patient can then reapply to ensure coverage over the entire surface of the nail. That is, the formulation is a film-forming formulation. The composition also acts as a barrier to protect the surface of the nail, preventing re-infection and sealing in the peptide.

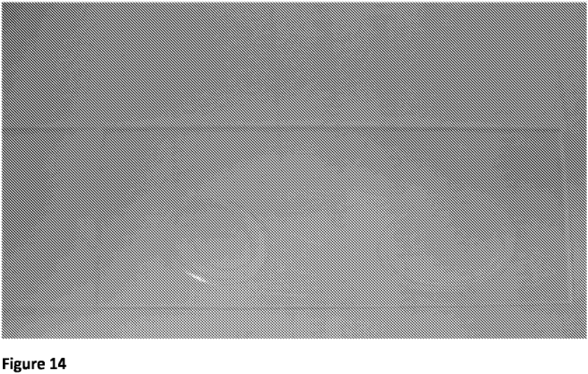

By film is meant a thin, visible, non-tacky layer formed on the surface of the nail. The drying times below relate to the time taken to form this film. An example of a film is provided in FIG. 14.

### Device

Typically, the topical application is applied using an applicator such as a dropper, pipette, sponge or brush.

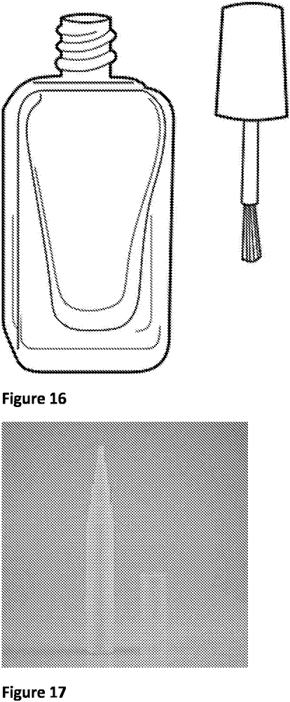

A device may be used to apply the composition to the nail. The device may for example be a nail pen (FIG. 17) or a bottle with brush applicator (FIG. 16).

The applicator for applying the treatment may be a sponge or a brush. Advantageously, use of an applicator permits a controlled amount of a composition to be laid down on the nail. Further advantageously, use of a sponge or brush permits the composition to be spread evenly, or approximately evenly, over the surface of the nail.

### Drying Time

Drying time or “drying over a period” as employed herein means the average time taken for a predetermined amount of the composition to dry on the nail and form a film. A dry composition is one that is dry to touch, that is, no tackiness remains to the touch. In general, when wearing nitrile gloves, the gloves do not stick to the nail if the nail is dry.

Drying time is affected by many factors, including but not limited to room temperature when applied, the amount applied, nail size, warmth of the nail (determined by the body surface temperature of the user), how well distributed the composition is on the nail. Drying time is typically assessed at a room temperature of approximately 19-22° C.

As employed herein a relatively low drying time means that a predetermined amount of the composition is dry to touch in less than about 35 minutes from application. For example, in a period of between 3 minutes to 35 minutes.

Predetermined amount as employed herein means about 10µl or less. That is, about 0.1 µl per 1 mm² of nail or less.

### Amount of Composition

As an example, the patient may paint the composition on to the nail once per day for 28 days using a 10% (w/v) solution of NP213. If 10 µl per nail is used, the calculation is as follows:

10% = 100 mg/ml = 1 mg in 10 µl, which equates to 28 mg per nail over the treatment course outlined above of 28 days.

For a larger toenail, more composition may be used, for example up to 150 µl per day. Use PEG 500-8000 has been found to be useful as a film-forming agent for nail compositions. Therefore, the use of PEG 500-8000 as a film forming agent in aqueous topical nail compositions is provided. This film-forming agent is particularly useful for protein containing nail compositions. This is because the normal nail lacquers used to form films are not suitable for proteins as they contain organic solvents. By using PEG instead, the protein maintains its efficacy. Additionally, PEG does not damage the nail as typical organic solvents do. Therefore, the advantageous effects of using PEG are: 1) better carrier for protein containing compositions; 2) ability to form a film which dries quickly; and 3) the avoidance of organic solvents which damage the nail.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

### EXAMPLES

### Example 1: Formulation of Peptide in Traditional Solvent-Based Nail Lacquers Compared With PEG

The inventors sought to find a peptide formulation which allowed the user to see where the peptide had been applied to; and maintained the efficacy of the peptide.

The activity of NP213 (cyclic Arginine 7-mer peptide: cyclic R-R-R-R-R-R-R, as a representative of the 4-15mers known to have anti-fungal activity, see Example 5) was tested in a selection of nail lacquers that had been used previously with other topical antifungals for the treatment of onychomycosis (those manufactured by Anacor and Roehm). These lacquers are film-forming allowing the user to see which areas have been covered by the anti-fungal treatment. However, NP213 was not effective in the known nail lacquers.

The inventors then moved away from these traditional lacquers and tested different formulations for NP213. It was found PEG maintained the efficacy of the cyclic peptide and allowed the formation of a film upon application. As explained above this usefully allows the user to see which areas have been treated with the peptide.

A comparison was carried out between the current nail lacquers used for topical treatment and PEG.

NP213 (5 and 10% w/v) was formulated in 50% (v/v) PEG4000.

Alternatively, NP213 was dissolved/suspended in the lacquer formulations at concentrations of 0, 5 or 10 % (w/v). 10 µl of each lacquer was applied to the base of a well in a flat-bottomed 96-well plate in triplicate wells and allowed to dry for 90 min.

An inoculum of 3 × 10³ cfu T. rubrum NCPF0118 was prepared in 1 × RPMI-1640 liquid medium and 200 µl was added to all wells containing lacquers and control wells containing no lacquer (growth control). An additional 3 wells per plate contained 200 µl RPMI-1640 liquid medium without inoculum to serve as sterility controls.

To each well was added a minimal volume of Alamar blue solution (0.0025% (w/v) final concentration). Antifungal activity was determined based on changes in metabolic activity (due to metabolism of the cell viability indicator Alamar blue). Metabolic activity was monitored by fluorescence (Ex 530 nm/Em 590 nm) every 24 h for up to 168 h.

The compositions of the lacquers were as follows:

| | | |
|---|---|---|
| 2d Lacquer | Anacor lacquer C | Roehm Lacquer 1: |
| 5.18% (w/v) Cellulose acetate butyrate | 55 - 65% (v/v) Ethanol | 0.15 g Triacetin |
| 51.14 - 61.14% (v/v) Acetone | 15% (v/v) Ethyl acetate | 0.28 g Urea |
| 1.55% (v/v) Acetyl tributylcitrate (tributylacetyl citrate) | 15% (w/v) Poly(vinylacetate) | 3.1 g 90% Ethanol |
| 32.12% (v/v) Ethanol | 5% (w/v) Dibutyl sebacate | 5.9 g Acetone |
| | | 0.5 g Cellulose acetate |

| Roehm Lacquer 2 | Roehm Lacquer 3 | Roehm Lacquer 4 : |
|---|---|---|
| 0.15 g 1,2-Propanediol | 0.15 g Dibutyl phthalate | 0.15 g Triacetin |
| 0.28 g Urea | 0.28 g Urea | 5.9 g 90% Ethanol |
| 3.1 g 90% Ethanol | 3.1 g 90% Ethanol | 5.9 g Acetone |
| 5.9 g Acetone | 5.9 g Acetone | 0.5 g Cellulose acetate |
| 0.5 g Cellulose acetate | 0.5 g Cellulose acetate | |
| Roehm Lacquer 5 | | |
| 0.15 g 1,2-Propanediol | | |
| 5.9 g 90% Ethanol | | |
| 5.9 g Acetone | | |
| 0.5 g Cellulose acetate | | |

### Results

When prepared in Roehm lacquer 1, the antifungal activity of 5% (w/v) NP213 and 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 1).

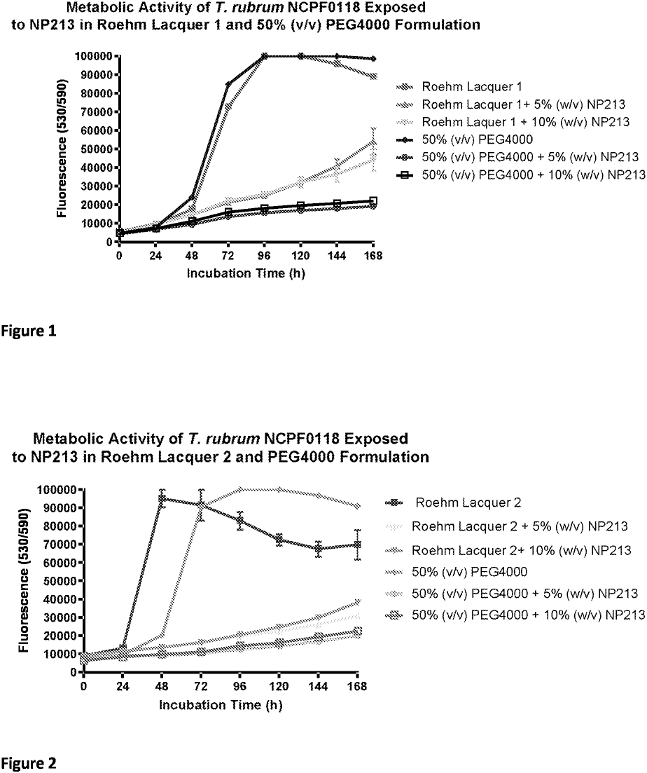

When prepared in Roehm lacquer 2, the antifungal activity of 5% (w/v) NP213 and 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 2).

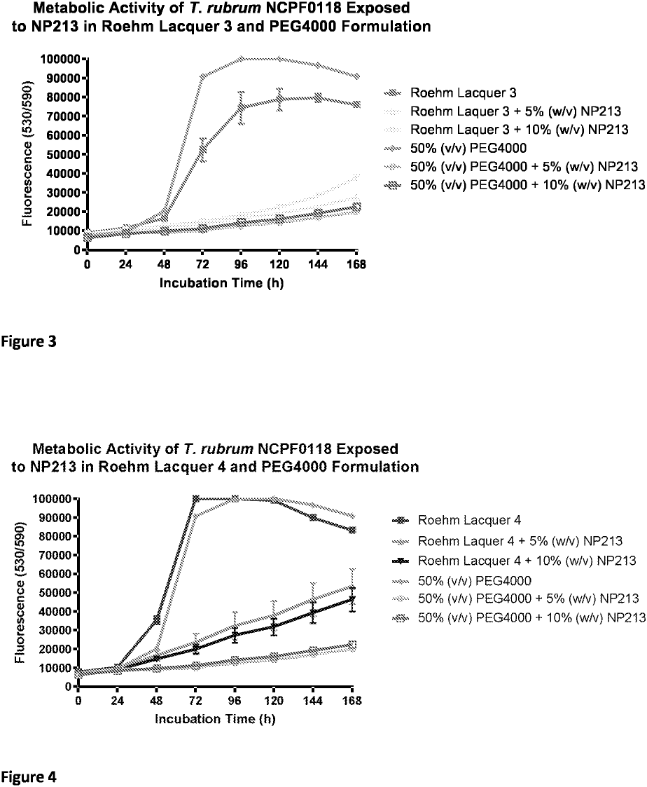

When prepared in Roehm lacquer 3, the antifungal activity of 5% (w/v) NP213 and 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 3).

When prepared in Roehm lacquer 4, the antifungal activity of 5% (w/v) NP213 and 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 4).

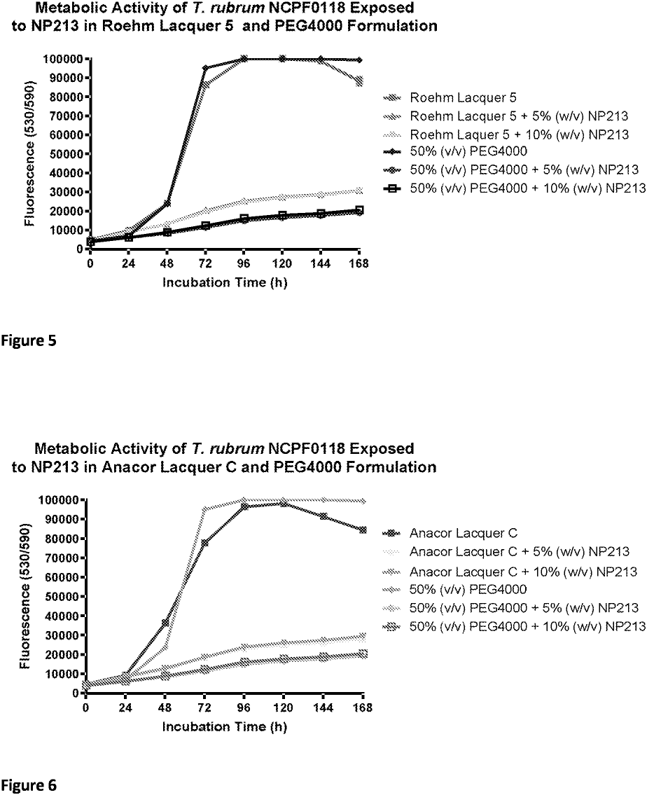

When prepared in Roehm lacquer 5, the antifungal activity of 5% (w/v) NP213 and 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 5).

When prepared in Anacor lacquer C, the antifungal activity of 5% (w/v) NP213 and 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 6).

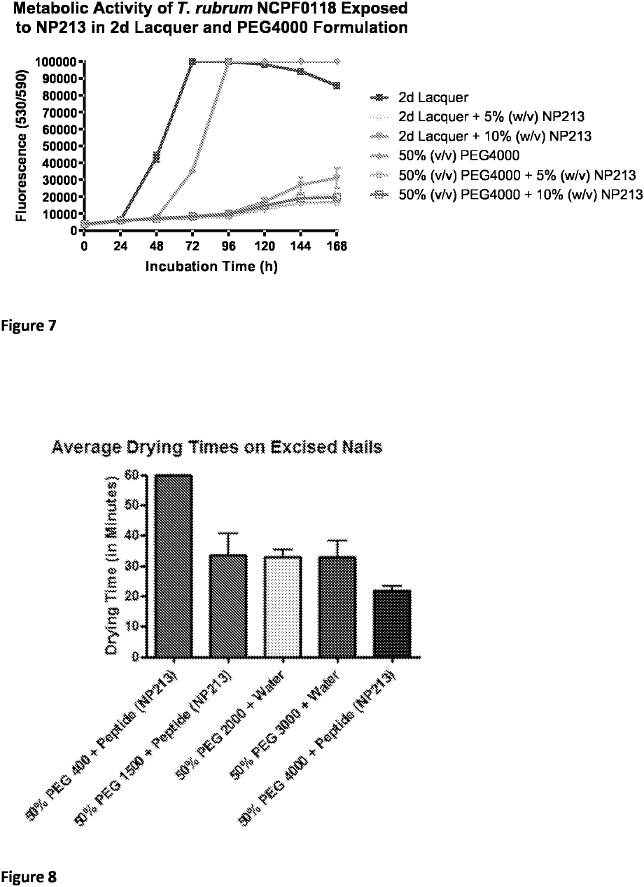

When prepared in 2d lacquer, the antifungal activity of 10% (w/v) NP213 against T. rubrum NCPF0118 was lower (as evidenced by higher metabolic activity) when compared to the activity of 5% (w/v) NP213 and 10% (w/v) NP213 when prepared in 50% (v/v) PEG4000 (FIG. 7).

### Results Summary

Water and PEG was found to be a superior formulation in maintaining the efficacy of the peptide.

None of the tested lacquer formulations were superior to that of water and 50% PEG4000 when assessing the antifungal activity of 5 or 10% (w/v) NP213 against T. rubrum NCPF0118.

As well as treating the fungal infection, the PEG composition hydrates the nail and improves its condition meaning the nail is more likely to recover from infection or trauma. This is in contrast to the currently used organic solvent lacquers which are not nail friendly.

### Example 2: Various PEG Solutions Provide Short Drying Times Suitable for Topical Nail Application

Peptide NP213 (a 7 mer cyclic polyarginine) was made up to 40% weight to volume solution with water. This 40% solution was then diluted to 20% solution using sdH₂O in a safety cabinet. Various mwt PEG solutions were used. PEG was melted in a microwave oven as follows:
PEG400 - already liquid (Sigma Cat No P3265 Lot 054k0063)
PEG1500 - flakes (Fluka Cat No 86101 Lot 1164516)
PEG2000 - flakes (Sigma Cat No 84797 Lot BCBD7108V)
PEG3000 - flakes (Sigma Cat No 81227 Lot BCBB2865)
PEG4000 - flakes (Fluka Cat No 95904 Lot 1206119)

250 µl of PEG and 250 µl of 20% peptide were added to give a 50% PEG, 10% peptide, 40% water solution immediately in 2 ml microcentrifuge tubes (rounded bottom). Using trimmed tips warmed to 30° C. in an incubator, 250 µl of PEG was added to the microcentrifuge tube followed by 250 µl of 20% peptide. Solution appearances were noted on cooling and following mixing by inverting solutions.

For the PEG +water (no peptide) 50% solutions were made up in 2 ml cryovials (0.5 ml PEG and 0.5 ml sdH₂O) and left overnight to check whether PEG came out of solution. The results were noted.
PEG400 - stayed in solution
PEG1500 - stayed in solution
PEG2000 - stayed in solution
PEG3000 - stayed in solution
PEG4000 - mostly stayed in solution

Toenails obtained by a podiatrist were thoroughly washed and cut into 4 nails of approximately equal size. The nails were put into a small petri dish in the safety cabinet. 10 µl of each solution was added to the nail and drying times noted at 22° C. on the bench. Results are shown below in Table 1 and in FIG. 8.

TABLE 1

| | | 50% PEG + 10% peptide + water | | | | 50% PEG + Water | | | |
|---|---|---|---|---|---|---|---|---|---|
| | Drying Time (in minutes) 3 replicates | | | Mean | Drying Time (in minutes) 3 or 4 replicates | | | | Mean |
| PEG 400 | >60 | >60 | >60 | > 60 | | | | | |
| PEG 1500 | 25 | 27.5 | 48 | 38 | | | | | |
| PEG 2000 | | | | | 26.5 | 34.5 | 38 | 33 | 33 |
| PEG 3000 | | | | | 26 | 22 | 37.5 | 46 | 33 |
| PEG 4000 | 19 | 21.5 | 24.5 | 21.5 | | | | | |

### Results Summary

The peptide formulation with PEG showed drying times suitable for topical nail application. The drying times all relate to the time taken to form a film. As explained above, a film is desirable as it allows the user to see where the product has been applied. Traditional lacquers supply this film, but were not compatible with the peptides as they inhibited antifungal activity. The inventors have found that PEG produces a film-forming composition and importantly is compatible with the cyclic peptides in that the antifungal activity of the peptides is maintained in PEG.

### Example 3 Long Term Stability of Various PEG and Peptide Formulations

### Materials

A range of PEG types, concentrations and peptide concentrations were assessed:

| 0% (w/v) Peptide: | 5% (w/v) Novexatin (7-mer cyclic arginine peptide) : | 7.5% (w/v) Novexatin: |
|---|---|---|
| 47.5, 50.0, 52.5 & 55.0% (v/v) PEG100 | 47.5, 50.0, 52.5 & 55.0% (v/v) | 47.5, 50.0, 52.5 & 55.0% (v/v) |
| 47.5, 50.0, 52.5 & 55.0% (v/v) PEG1500 | PEG1000 | PEG1000 |
| 47.5, 50.0, 52.5 & 55.0% (v/v) PEG2000 | 47.5, 50.0, 52.5 & 55.0% (v/v) | 47.5, 50.0, 52.5 & 55.0% (v/v) |
| 47.5, 50.0, 52.5 & 55.0% (v/v) PEG3000 | PEG4000 | PEG4000 |
| 47.5, 50.0, 52.5 & 55.0% (v/v) PEG4000 | 47.5, 50.0, 52.5 & 55.0% (v/v) | 47.5, 50.0, 52.5 & 55.0% (v/v) |
| 47.5, 50.0, 52.5 & 55.0% (v/v) PEG8000 | PEG8000 | PEG8000 |
| 10% (w/v) Novexatin: | 12.5% (w/v) Novexatin: | 15% (w/v) Novexatin: |
| 47.5, 50.0, 52.5 & 55.0% (v/v) | 47.5, 50.0, 52.5 & 55.0% (v/v) | 47.5, 50.0, 52.5 & 55.0% (v/v) |
| PEG1000 | PEG1000 | PEG1000 |
| 47.5, 50.0, 52.5 & 55.0% (v/v) | 47.5, 50.0, 52.5 & 55.0% (v/v) | 47.5, 50.0, 52.5 & 55.0% (v/v) |
| PEG4000 | PEG4000 | PEG4000 |
| 47.5, 50.0, 52.5 & 55.0% (v/v) | 47.5, 50.0, 52.5 & 55.0% (v/v) | 47.5, 50.0, 52.5 & 55.0% (v/v) |
| PEG8000 | PEG8000 | PEG8000 |

Formulations were prepared in clear, graduated, screw capped glass vials and were assessed for physical appearance on a daily/weekly basis.
PEG1000: Thermo-Fisher; Cat no. 10752621; Lot no. A0369980
PEG1500: Sigma-Aldrich; Cat no. 86101; Lot no. 1164516
PEG2000: Sigma-Aldrich; Cat no. 84797; Lot no. BCBD7108V
PEG3000: Sigma-Aldrich; Cat no. 81227; Lot no. BCBB2865
PEG4000: Sigma-Aldrich; Cat no. 95904; Lot no. 1206119
PEG8000: Melford Laboratories Ltd; Cat no. P0808; Lot no. 19659
Novexatin (also called NP213 above, a 7 mer cyclic polyarginine): Polypeptide Laboratories; Lot no. GAP 10149; 97.93% purity; 65.10% peptide
Graduated Screw Top Vial (solid cap/Teflon liner): Sigma-Aldrich; Cat no. 27505-U; Lot no. 78573

### Methods

All glass and plastic-ware was pre-heated to 37° C. before starting the formulation work to ensure and facilitate accurate dispensing of PEG solutions.

Solutions of NP213 were prepared in sdH₂O (18 MΩ) at a concentration of 400 g/L and pre-warmed to 37° C. immediately prior to use in formulation preparation.

Solid PEG flakes/crystals were melted in glass Universal bottles in a microwave oven on low power until a clear solution was obtained. Immediately, the appropriate volume of PEG was transferred to 2 ml glass vials and the final volume made up to 1 ml with pre-warmed sdH₂O (18 MΩ) and/or concentrated peptide solution. Formulations were mixed by inversion to ensure homogeneity.

Formulations were stored at room temperature (18 - 20° C.) in the dark and assessed after 2 h and on a daily basis for the first few weeks, and then on a weekly basis for an indefinite period. Formulation appearance and ambient temperature were recorded in Microsoft Excel.

### Results

### 0% (W/V) Peptide

47.5, 50.0, 52.5 & 55.0% (v/v) PEG1000
47.5, 50.0, 52.5 & 55.0% (v/v) PEG1500
47.5, 50.0, 52.5 & 55.0% (v/v) PEG2000
47.5, 50.0, 52.5 & 55.0% (v/v) PEG3000
47.5, 50.0, 52.5 & 55.0% (v/v) PEG4000
47.5, 50.0, 52.5 & 55.0% (v/v) PEG8000

The PEG1000, PEG1500 and PEG2000 solutions remained clear and non-viscous for up to 33 weeks. The PEG3000 solution was initially clear and non-viscous, but became viscous after 8 - 9 d and remained the same for at least 33 weeks. The PEG4000 and PEG8000 solutions were clear and viscous for ≥48 weeks. This experiment was terminated after 48 weeks.

### Novexatin Results

Novexatin (NP213) formulations were prepared in the PEG1000, PEG4000 and PEG8000 solutions at 5.0, 7.5, 10.0, 12.5 and 15.0% (w/v) peptide.

PEG1000/Novexatin formulations remained as clear, non-viscous solutions for at least 34 weeks for 15.0% (w/v) NP213 and at least 37 weeks for all other NP213 concentrations.

PEG4000/Novexatin formulations containing 5.0 and 7.5% (w/v) NP213 remained as clear, viscous solutions for at least 31 weeks. PEG4000/Novexatin formulations containing 10.0 and 12.5% (w/v) NP213 remained as clear, viscous solutions for at least 31 weeks, with the exception of the solutions containing 52.5% (v/v) PEG. PEG4000/Novexatin formulations containing 10.0 and 12.5% (w/v) NP213 and containing 52.5% (v/v) PEG contained small white crystals (NP213, not PEG) on immediate preparation, but after 24 h incubation at room temperature the crystals had dissolved into clear, viscous solutions and remained so for at least 31 weeks. PEG4000/Novexatin formulations containing 15.0% (w/v) NP213 and containing 52.5% (v/v) PEG contained small white crystals (NP213, not PEG) on immediate preparation, but after 48 h incubation at room temperature the crystals had dissolved into clear, viscous solutions for 50.0 and 52.5% (v/v) PEG and remained so for at least 28 weeks.

PEG8000/Novexatin formulations containing 5.0 and 7.5% (w/v) NP213 remained as clear, viscous solutions for at least 31 weeks. PEG8000/Novexatin solutions containing 10.0% NP213 remained as clear, viscous solutions for at least 31 weeks, with the exception of the solutions containing 52.5 and 55.0% (v/v) PEG. PEG8000/Novexatin formulations containing 10.0% (w/v) NP213 and containing 52.5 or 55.0 % (v/v) PEG contained small white crystals (NP213, not PEG) on immediate preparation, but after 24 h incubation at room temperature the crystals had dissolved into clear, viscous solutions and remained so for at least 31 weeks. PEG8000/Novexatin solutions containing 12.5% NP213 and 47.5% (v/v) PEG8000 remained as clear, viscous solutions for at least 31 weeks. PEG8000/Novexatin solutions containing 12.5% NP213 and 50.0 or 52.5% (v/v) PEG contained small white crystals (NP213, not PEG) on immediate preparation, but after 24 h incubation at room temperature the crystals had dissolved into clear, viscous solutions and remained so for at least 31 weeks.

PEG8000/Novexatin solutions containing 15.0% NP213 and 47.5% or 50.0% (v/v) PEG8000 remained as clear, viscous solutions for at least 32 weeks. The PEG8000/Novexatin solution containing 52.5% (v/v) PEG and 15.0% (w/v) NP213 contained small white crystals (NP213, not PEG) on immediate preparation and remained for 37 d, whereupon they dissolved, leaving a clear, viscous solution that remained for at least a further 24 weeks.

### Summary

All PEG formulations prepared without dissolved peptide remained as clear solutions for at least 48 weeks. All PEG formulations containing PEG1000 (47.5 - 55.0% (v/v)) and Novexatin (5.0 -15.0% (w/v)) were clear, non-viscous solutions and remained so for at least 32 - 34 weeks.

All PEG formulations containing PEG4000 (47.5 - 55.0% (v/v)) and Novexatin (5.0 - 15.0% (w/v)) were clear solutions and remained so for at least 19 weeks.

-All PEG formulations containing PEG8000 (47.5 - 55.0% (v/v)) and Novexatin (5.0- 15.0% (w/v)) were clear solutions and remained so for at least 19 weeks.

### Example 4: EDTA and Other Protease Inhibitors Also Have Antifungal Activity Against Various Nail Fungal Pathogens: The Effect of Protease Inhibitors on the Minimum Inhibitory Concentration (MIC) and Minimum Fungicidal Concentration (MFC) of NP213 (Cyclic Polyarginine Peptide, i.e. Cyclic R-R-R-R-R-R-R)

Antifungal susceptibility testing to determine the MIC was conducted by the broth microdilution procedure for filamentous fungi (M38-A2) (Clinical & Laboratory Standards Institute (CLSI). 2008. Reference Method for Broth Dilution Antifungal Susceptibility Testing of Filamentous Fungi; Approved Standard - Second Edition (M38-A2). Clinical and Laboratory Standards Institute, Wayne, PA). This experiment was carried out in triplicate with triplicate samples in each experiment. The proteolytic stability of NP213 was assessed by determining NP213 MIC as described above versus T. rubrum NCPF0118 and T. interdigitale NCPF0335 in the presence of the following protease inhibitors; serine proteases (1 mM phenylmethylsulfonyl fluoride; PMSF), metalloproteases (100 µM ethylenediaminetetraacetic acid; EDTA), aspartyl proteases (1 mg/L Pepstatin A) and cysteine proteases (10 µM trans-epoxysuccinyl-L-leucylamido(4-guanidino)butane; E-64). Protease inhibitors at these concentrations had no effect on the growth of either T. rubrum NCPF0118 or T. interdigitale NCPF0335.

### Modified Antifungal Susceptibility Testing Procedure

Antifungal susceptibility testing was carried out as described above, but with the following modifications. RPMI-1640 medium was substituted for 0.5% (w/v) powdered nail, skin keratin or sheep’s wool keratin suspensions in 10 mM sodium phosphate buffer, pH 7.0 containing 0.0025% (w/v) AlamarBlue™ (ThermoFisher Scientific, UK). Human nails were obtained from a NHS podiatrist (NHS Grampian), following appropriate ethical approval (REC reference: 05/S0801/115), from donors following nail avulsion due to ingrown toenails. All nails were disease-free, based on the podiatrist’s diagnosis. Nails were cut into small fragments and ground to a fine powder in liquid nitrogen in a mortar and pestle. Following this, powdered nail was passed through a fine-meshed sieve and sieved nail powder was used to prepare suspensions in 20 mM sodium phosphate buffer, pH 7.0. Nail powder suspensions were sterilised by autoclaving for 20 min at 121° C. Sheep’s wool and human skin keratin were obtained from ABCR GmbH.

Metabolic activity was monitored by fluorescence (Ex 530 nm/Em 590 nm) every 24 h for up to 216 h, or until high metabolic activity (100000 U) was observed in inoculated controls in the absence of any antifungal agent. As a control, antimicrobial susceptibility testing was conducted in RPMI-1640 medium and MICs were determined by measuring both optical density and metabolic activity. Sodium phosphate buffer, pH 7.0, alone did not support dermatophyte growth (data not shown). This experiment was carried out in triplicate with triplicate samples in each experiment.

### Results

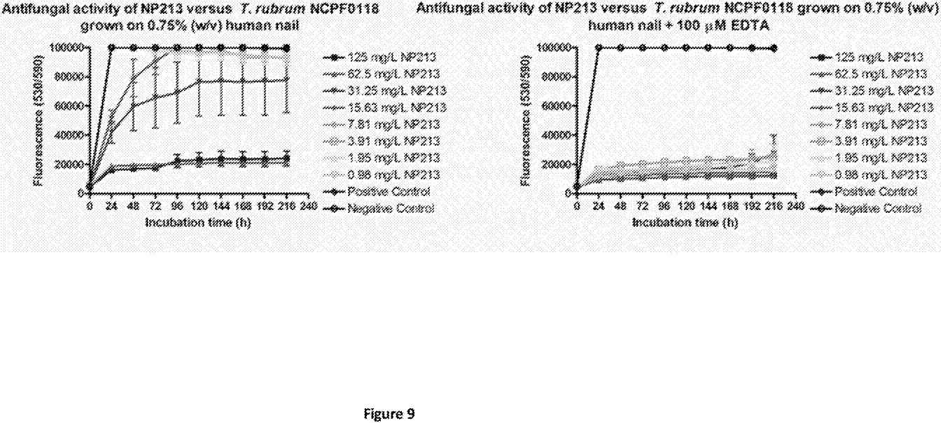

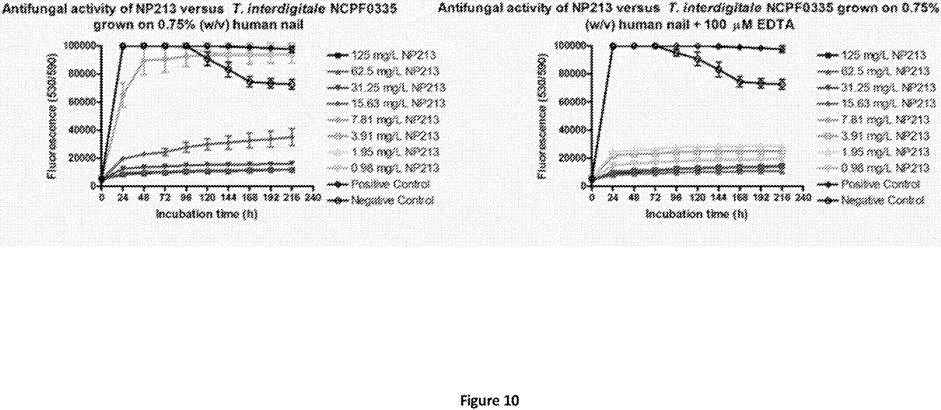

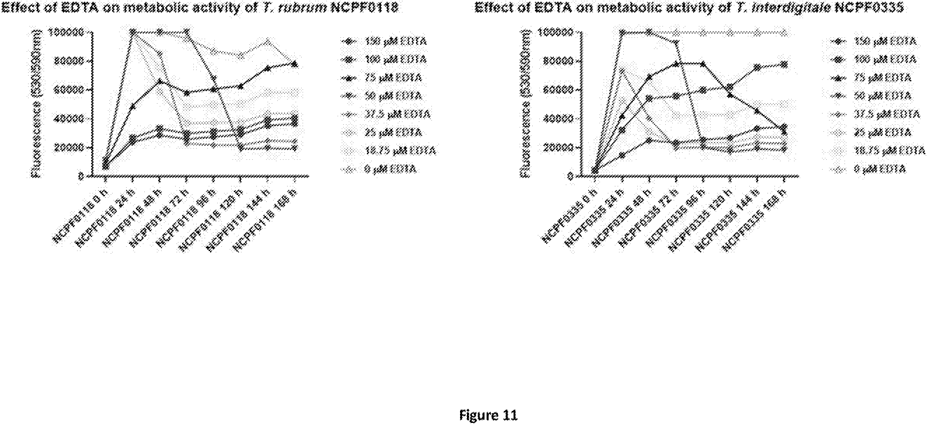

The data is shown in FIGS. 9-11 and in Table 2 below.

TABLE 2

| T. rubrum NCPF0118 | MIC (mg/L) @ 216 h |
|---|---|
| RPMI-1640 | 1000 |
| 0.75% (w/v) Nail | 62.5 |
| 0.75% (w/v) Nail + 100 µM EDTA | 0.98 |
| 0.75% (w/v) Nail + 50 µM EDTA | 15.63 |
| 0.75% (w/v) Nail + 50 µM Bestatin | 62.5 |
| RPMI-1640 | 500 |
| 0.75% (w/v) Nail | 62.5 |
| 0.75% (w/v) Nail + 100 µM EDTA | 7.81 |
| 0.75% (w/v) Nail + 50 µM EDTA | 15.63 |
| 0.75% (w/v) Nail + 50 µM Bestatin | 125 |

TABLE 3

Effect of other protease inhibitors on the antifungal activity of NP213 against the dermatophytes
MIC (mg/L)

| | T.rubrum NCPF0118 | T.interdigitale NCPF0335 |
|---|---|---|
| NP213 | 1000 | 500 |
| NP213 + Metalloprotease Inhibitor | 500 | 250 |
| NP213 + Serine protease Inhibitor | 500 | 500 |
| NP213 + Cysteine protease Inhibitor | 1000 | 500 |
| NP213 + Aspartyl protease Inhibitor | 1000 | 500 |

T. rubrum NCPF0118 and T. interdigitale NCPF0335 in RPMI-1640 liquid medium. MIC was determined by the broth microdilution procedure (CLSI, 2008). Metalloprotease inhibitor -100 µM EDTA; Serine protease inhibitor - 1 mg/L PMSF; Cysteine protease inhibitor -10 µM E64; Aspartyl protease inhibitor - 1 mg/L pepstatin. Protease inhibitors did not inhibit growth of either isolate in RPMI-1640 liquid medium at the concentrations tested.

### Results Summary

Metalloprotease inhibitors including EDTA and Bestatin enhanced the anti-fungal effect of the cyclic peptide.

### Example 5: Peptides Have Antifungal Activity Across the Amino Acid Range Claimed

Antifungal susceptibility testing was conducted by the broth microdilution procedure as described in CLSI Approved Standard M38-A2. All peptides were dissolved in sterile deionised water (40 mg/ml) and stored at -20° C. until required. All experiments were carried out in triplicate, with triplicate samples in each experiment.

TABLE 4

Antifungal activity of cyclic arginine peptides against T. rubrum NCPF0118 and T. rubrum NCPF0335
MIC (mg/L)

| Peptide | T.rubrum NCPF0118 | T. interdigitale NPCF0335 |
|---|---|---|
| NP201 (cyclic-5R) | 500 | 1000 |
| NP202 (cyclic 11 R) | 250 | 15.6 |
| NP203 (cyclic 15R) | 62.5 | 3.9 |
| NP213 (cyclic-7R) | 1000 | 500 |

### Results Summary

Cyclic peptides across the range claimed show antifungal activity across different species of fungus.

### Example 6: Substitutions Retain Antifungal Activity

Antifungal susceptibility testing was conducted by the broth microdilution procedure as described in CLSI Approved Standard M38-A2. All peptides were dissolved in sterile deionised water (40 mg/ml) and stored at -20° C. until required. All experiments were carried out in triplicate, with triplicate samples in each experiment.

### Results Summary

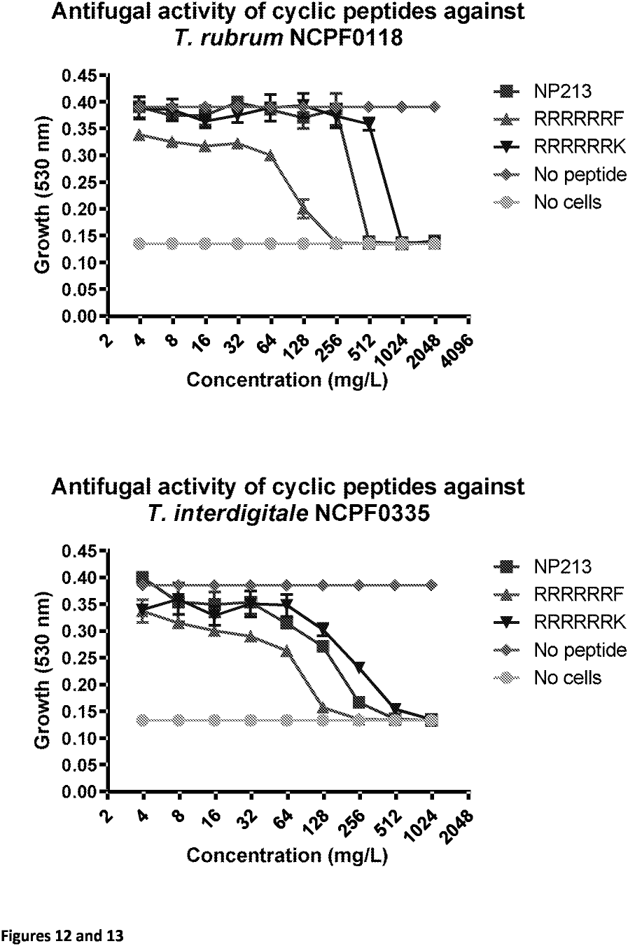

The results are shown in FIGS. 12 and 13. Substitutions of R and F do not adversely effect the activity of the cyclic peptide.

### Example 7: Faster Treatment With Cyclic Peptide Than Currently Used Antifungal Treatments the Time Taken for the Cyclic Peptides to Provide an Antifungal Effect Was Tested

An experimental model of dermatophyte nail infection was optimized to simulate onychomycosis under laboratory conditions and to test the antifungal efficacy of NP213. As an inert support and to provide a moist atmosphere, sterile water agar plates were prepared in 90-mm Petri dishes (1.5% (w/v) agar in sterile-deionised water). Petri dishes were divided into three sections, and individual pieces of sterile silicone rubber tubing (1-cm diameter; 3-mm height) were placed in each section, allowing the investigation of three nail fragments per plate. This prevented direct contact between the nails and the agar surface or the lid of the Petri dish. Such a setup was necessary because agar contains components that can bind to cationic peptides, including NP213, inhibiting their diffusion and reducing activity. Silicon tubing is permeable to oxygen and carbon dioxide, and its use ensured oxygen access to support fungal growth. Its diameter was sufficiently small to stably support the nail fragments. Uninfected human nails (sourced under ethical approval) were cleaned of all residual skin and debris before cutting to size (approximately four fragments were obtained from an adult male great toenail), and their surface area and thickness were determined using a micrometer. Nail fragments were submerged in 10 ml of deionised water and sterilized by autoclaving at 134° C. for 20 min. The samples were then mounted with the ventral (nail bed side, concave) surface facing upward on the silicon tubing rings. Nail samples were allocated to provide nails of similar average size and thickness for each treatment. Nails were left to dry and incubated to ensure sterility for 7 days in a humid atmosphere at 30° C. Each nail fragment was inoculated on its ventral surface with a spore suspension containing approximately 2 × 107 spores of Trichophyton spp. in sterile 0.15 M NaCl (0.01 ml spore suspension per cm2 nail surface area). Experiment one was carried out with Trichophyton rubrum NCPF0118. Experiment two was carried out with Trichophyton rubrum ATCC-MYA-4438. Experiment three was carried out with Trichophyton rubrum S52d0. Petri dishes were incubated at 30° C. inside a sealed plastic box containing an sterile deionised water reservoir to avoid dehydration, for 14 days, at which point hyphal growth was clearly visible on the entire surface of the nail fragment. Nails within the same Petri dish were always exposed to the same fungal inoculum and treatment, mitigating the risk of cross-contamination.

All nail fragments were treated by application of NP213 or controls to the dorsal side of the nail. The dorsal (convex) aspect of the nail is that normally exposed to the environment. Antifungal agents were applied (0.01 ml/cm2 nail surface area) daily and distributed over the surface of the nail area with a sterile nail lacquer brush applicator. After 7, 14 or 28 days of daily treatment with NP213, nail fragments were transferred to sterile 2.0-ml microcentrifuge tubes containing 1 ml of SAB broth containing 6% (w/v) polyanetholesulfonic acid (PASA) to neutralize residual NP213 and with 100 mg of sterile 0.5-mm-diameter glass beads. The contents of the tubes were mixed thoroughly for 1 min using a bead-beater to release fungal cells from the nail matrix for enumeration, and stepwise 10-fold dilutions were prepared in Sabouraud dextrose broth containing 3% (w/v) PASA. Samples were plated on potato dextrose agar and incubated at 30° C. for 14 days, and the colonies were counted.

### Results Summary

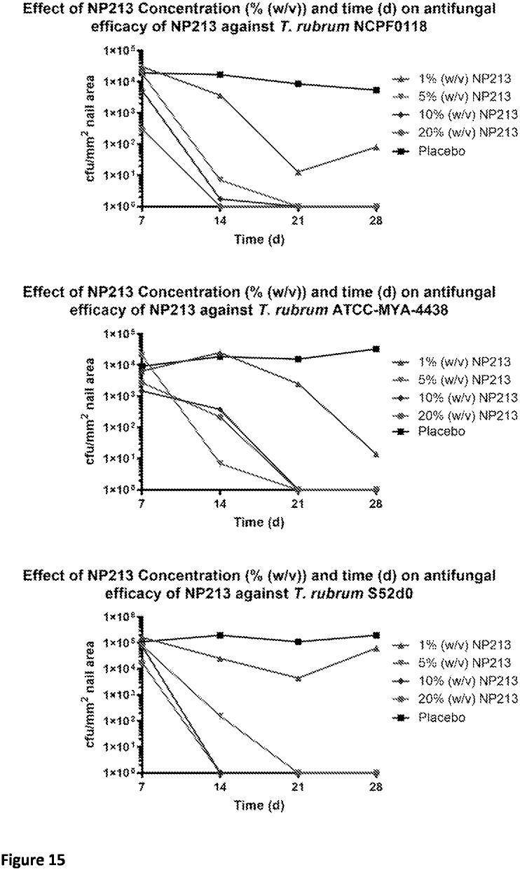

The results are shown in FIG. 15. These show that antifungal effects are seen within 11 days of beginning treatment. This is in contrast to the current antifungal treatments which often require 48 weeks of treatment at least before any improvement in the nail is seen.

1. An aqueous composition for topical nail application, the composition comprising:
a) a protein; and
b) polyethylene glycol (PEG) 500 — PEG 8000.

2. The aqueous composition of claim 1, wherein the composition is a film-forming composition.

3. The aqueous composition as in claims 1-2, wherein the PEG has an average molecular weight of between 1000 and 6000, optionally wherein the PEG has an average molecular weight of between 4000 and 6000.

4. The aqueous composition as in any of the preceding claims, wherein the composition comprises between 10 and 80% v/v PEG, optionally wherein the composition comprises between about 40% and 60% v/v PEG.

5. The aqueous composition of any of claims 1-4, wherein the protein is collagen and/or keratin.

6. The aqueous composition of claim 5, wherein the composition comprises a nail enhancer, optionally wherein the nail enhancer is biotin.

7. A cosmetic method for treatment of a nail, the method comprising applying the aqueous composition of any of claims 1-6 to the nail.

8. The method of claim 7, wherein the composition forms a film on the nail.

9. The aqueous composition of any of claims 1-6, wherein the protein is an antifungal cyclic peptide comprising 4-15 arginines subject to 0 or 1 substitutions in the peptide.

10. The aqueous composition as claimed in claim 9, additionally comprising any one or more of the following:
a) a protease inhibitor, optionally a metallopeptidase inhibitor; b) keratin; or c) collagen.

11. An aqueous composition comprising:
a) an antifungal cyclic peptide comprising 4-15 arginines subject to 0 or 1 substitutions in the peptide; and
b) a protease inhibitor, optionally a metallopeptidase inhibitor.

12. The aqueous composition as claimed in claim 10 or 11 wherein the metallopeptidase inhibitor is an endoprotease inhibitor, optionally
a) an inhibitor of the fungalysin family (M36); or
b) ethylenediaminetetraacetic acid (EDTA).

13. The aqueous composition according to claims 9-12 wherein the peptide is present at about 1% w/v to 20% w/v of the composition.

14. The aqueous composition as claimed in claims 9-13, wherein the peptide comprises 5 to 9 arginine residues.

15. The aqueous composition according to any of claims 9-14, wherein the antifungal cyclic peptide is an acid salt form, optionally acetate.

16. A pharmaceutical composition comprising the aqueous composition of claims 9-15.

17. The aqueous composition according to any of claims 9-16 for use as a medicament.

18. The aqueous composition according to any of claim 17 for use in the treatment or prevention of a fungal nail infection wherein the composition is applied topically.

19. The aqueous composition for use according to claim 18, wherein the fungal nail infection is caused by a dermatophyte.

20. The aqueous composition for use according to claim 18 or 19 wherein the composition is administered to the surface of the nail on a daily basis.

21. The aqueous composition for use according to claim 20, wherein the composition is applied daily over a period from 7-28 days.

22. A composition comprising an antifungal cyclic peptide comprising 4-15 arginines subject to 0 or 1 substitutions in the peptide, for use in the treatment or prevention of a fungal nail infection, wherein the composition is applied to the surface of the nail daily over a period from 7-28 days.

23. The composition for use according to claims 20-22, wherein the treatment is repeated every 3-12 months.

24. The composition for use according to claim 22-23, wherein the antifungal cyclic peptide is an acid salt form, optionally acetate.

25. The composition for use of claims 22-24, wherein the antifungal cyclic peptide is present at about 1% w/v to 20% w/v of the composition.

26. The composition for use according to claims 22-23 wherein the composition is any of the aqueous compositions of claims 9-16.

27. A device for applying a composition to a nail, wherein the device comprises the composition of claims 1-6 or 9-16.

28. The device of claim 27, wherein the device comprises a brush to apply the composition to the nail, optionally wherein the device is a nail pen or a bottle and brush.

29. Use of PEG 500-8000 in a topical nail composition to form a film on a nail, wherein the topical nail composition is an aqueous composition comprising a protein.
